(19) United States
(12) Patent Application Publication
(10) Pub. No.: US 2013/0041278 A1
(43) Pub. Date: Feb. 14, 2013

(54) METHOD FOR DIAGNOSIS OF DISEASES VIA ELECTRONIC STETHOSCOPES

(71) Applicants: Mingsian R. BAI, Hsinchu City (TW); Chun-Ching Wu, Hsinchu City (TW); Wan-Chih Chao, Hsinchu City (TW); Lu-Cheng Kuo, Taipei city (TW); Pen-Chung Yew, Taipei city (TW); Hsin-Min Wang, Taipei city (TW); Fu Chang, Taipei city (TW); Wen-Liang Hwang, Taipei city (TW)

(72) Inventors: Mingsian R. BAI, Hsinchu City (TW); Chun-Ching Wu, Hsinchu City (TW); Wan-Chih Chao, Hsinchu City (TW); Lu-Cheng Kuo, Taipei city (TW); Pen-Chung Yew, Taipei city (TW); Hsin-Min Wang, Taipei city (TW); Fu Chang, Taipei city (TW); Wen-Liang Hwang, Taipei city (TW)

(21) Appl. No.: 13/298,980

(22) Filed: Nov. 17, 2011

(30) Foreign Application Priority Data
Aug. 11, 2011 (TW) 100128638

(51) Int. Cl. A61B 7/04

(52) U.S. Cl. 600/529

(57) ABSTRACT

A method for diagnosis of diseases adopted on an electronic stethoscope which includes at least two sound receiving portions, a noise control portion, a processing portion, a data portion and an output portion. The method includes: first, the sound receiving portions receive sound signals issued from a patient's lungs included external noises; next, the sound signals are sent to the noise control portion which eliminates the external noise, and the processing portion to be overlapped and intensified; then characteristic values are retrieved from the sound signals to be compared with disease sound signal data in the data portion; finally the output portion outputs a diseases judgment result. Thus the electronic stethoscope can perform automatic interpretation of diseases to reduce human erroneous diagnostic judgment. Users also can get preliminary understanding of their body conditions when doctors are absent.

### FIELD OF THE INVENTION

The present invention relates to an electronic stethoscope and particularly to a method for diagnosis of diseases adopted for use on an electronic stethoscope.

### BACKGROUND OF THE INVENTION

Stethoscope is an important device for people in medical fields in performing medical tasks. Through sound amplifying function of the stethoscope doctors can understand activity conditions of a patient's internal organs, then incorporate with professional knowledge and experiences to make an preliminary diagnosis and take desirable treatments. Most conventional stethoscopes are mechanical types, such as U.S. Pat. Nos. 5,945,640 and 6,725,966. The mechanical stethoscope receives sound issued from a patient's organs via a head piece in contact with the patient's body. The sound passes through a pliable Y-shaped hose and is channeled into doctor's ears via an ear tip.

During sound transmission through the pliable lengthy hose, resonance frequently takes places and results in sound distortion. Moreover, during capturing the sound the stethoscope is being moved around to form friction with patient's clothes or doctor's fingers to generate noises which also are resonated and amplified. All that creates confusion or interference of the sound to become less clear, and could affect accuracy of doctor's judgment. Hence doctor has to pay a great attention to hear small sound signals of various body portions of the patient. To make accurate judgment relies on many years of accumulated experiences of the doctor. It takes huge investments in manpower and efforts to acquire the needed experiences. Moreover, doctor could also make erroneous judgment due to personal factors and result in medical malpractice disputes or claims. In addition, the conventional stethoscope can only allow the doctor to hear sound issued from patient's organs onsite and cannot save the sound file. After the patient has been treated for a period of time, and another diagnosis has to be made by hearing the sound from the patient's body, no comparison of the sound can be made with the prior treatment. This makes judgment of treatment efficacy more difficult. Moreover, the patient cannot hear the sound issued from his/her organs.

In view of the aforesaid disadvantages, electronic stethoscope has been developed, such as R.O.C. Pat. No. 558434 entitled “Electronic stethoscope and method of same” which includes a digital signal processor (DSP) to select bandwidth and provide advanced noise process to enhance sound signal capturing quality. R.O.C. Pat. No. M351067 entitled “Improved stethoscope MP3” records and saves captured signals in a MP3 module to facilitate doctor's diagnosis. The aforesaid electronic stethoscopes mostly provide merely noise elimination, sound receiving and recording functions. While they provide play back function to allow comparison before and after treatments, interpretation of the sound signal data and diseases still relies on accumulation of experiences of doctors that take many years. The human cost is very high and diagnostic accuracy is prone to be affected by human factors. Thus erroneous interpretations cannot be fully ruled out, and medical malpractice disputes and claims could occur.

### SUMMARY OF THE INVENTION

The primary object of the present invention is to solve the problems of the conventional stethoscope that cannot make judgment based on the heard sound signals and is highly relied on doctor's diagnosis. Another object of the invention is to enhance the signal intensity of the sound signals captured by the stethoscope to improve disease judgment accuracy.

To achieve the foregoing objects, the present invention provides a method for diagnosis of diseases adopted for use on an electronic stethoscope. The electronic stethoscope includes at least two sound receiving portions, a noise control portion, a processing portion, a data portion and an output portion. The noise control portion is connected to the sound receiving portions and the processing portion. The data portion is connected to the processing portion and stores disease sound signal data. The output portion is connected to the processing portion. The method includes the steps as follows:

Step S1: The sound receiving portions receive multiple sound signals issued from a patient's lungs included external noises, and output to the noise control portion;

Step S2: The noise control portion eliminates the external noises and sends the sound signals to the processing portion;

Step S3: The sound signals are overlapped through the processing portion to form an intensified sound signal including N1 characteristic values.

Step S4: Retrieve N2 characteristic values that have greater impact from the N1 characteristic values of the intensified sound signal, wherein N2 is smaller than N1; and

Step S5: Judge the N2 characteristic values matching disease sound signal data in the data portion, and the output portion outputs a disease judgment result.

By means of the method of the invention set forth above, the sound receiving portions can receive sound signals issued from patient's lungs, the noise control portion can eliminate the external noises in the sound signals, and the processing portion can overlap the sound signals and get the characteristic values of greater impact from the sound signals, and compare the characteristic values with the disease sound signal data in the data portion to output the judgment result of the disease. Thus automatic interpretation of diseases can be achieved via the electronic stethoscope to reduce human's erroneous interpretation made by human diagnosis. Moreover, users can use the electronic stethoscope by themselves to get preliminary understanding of their body conditions before asking advices of the doctor, thus enhancing accuracy of diseases interpretation.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of the structure of a first embodiment of the electronic stethoscope of the invention.

FIG. 2 is a flowchart of the first embodiment of the invention.

FIG. 3 is a flowchart of establishing disease sound signal data according to the first embodiment of the invention.

### DETAILED DESCRIPTION OF THE PREFERRED EMBODIMENTS

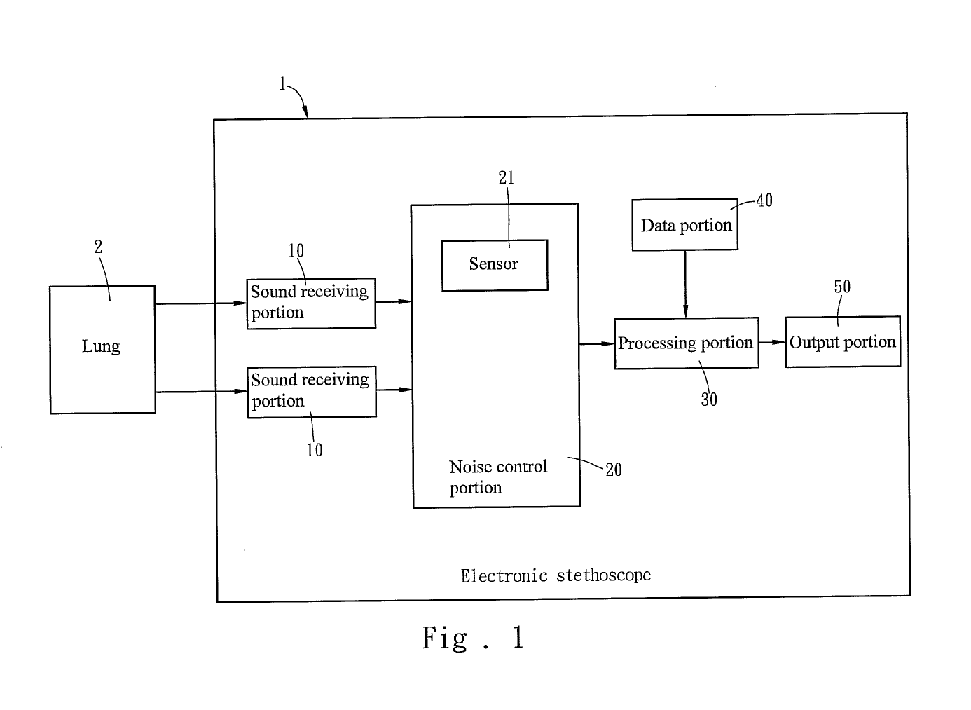

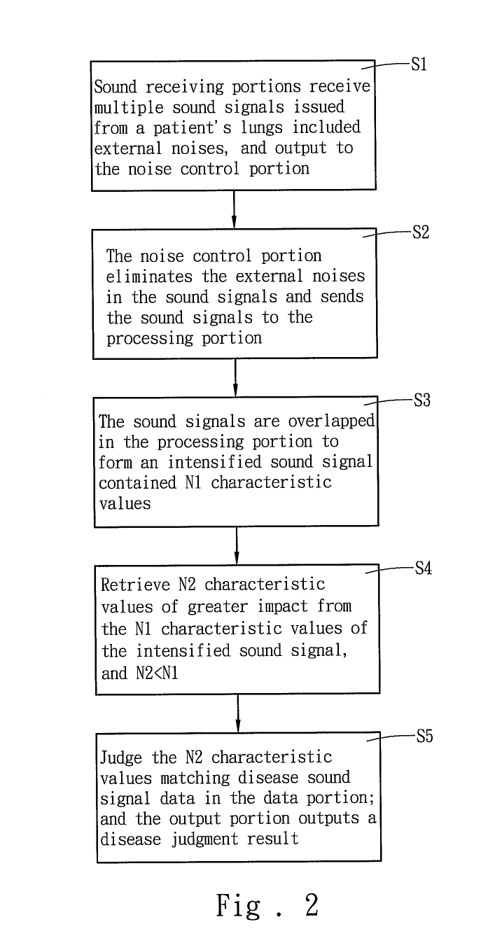

Please refer to FIGS. 1 and 2 for a first embodiment of the electronic stethoscope of the invention and the flowchart of the method for diagnosis of diseases adopted for the first embodiment. The electronic stethoscope 1 includes at least two sound receiving portions 10, a noise control portion 20, a processing portion 30, a data portion 40 and an output portion 50. The noise control portion 20 is connected to the sound receiving portions 10 and the processing portion 30. The data portion 40 is connected to the processing portion 30 and stores disease sound signal data. The output portion 50 is connected to the processing portion 30. The method includes the steps as follows:

Step S1: The sound receiving portions 10 receive multiple sound signals issued from a patient's lungs 2 included external noises, and output to the noise control portion 20. In this embodiment the sound receiving portions 10 include at least two sets of microphones made via micro-electromechanical processes. The sound receiving portions 10 are arranged in an array fashion to collect multiple sound signals at different locations spaced from the patient's lungs included external noises, then send the sound signals to the noise control portion 20.

Step S2: The noise control portion 20 eliminates the external noises in the sound signals and sends the resulting sound signals to the processing portion 30. In this embodiment the noise control portion 20 includes a sensor 21 to detect the external noises not issued from the patient's lungs 2 and generate a counter-noise signal according to the external noises to offset the external noises. Then the noise control portion 20 sends the sound signals without the external noises to the processing portion 30.

Step S3: The sound signals are overlapped in the processing portion 30 to form an intensified sound signal containing N1 characteristic values. In this embodiment the processing portion 30 receives the sound signals and processes Time Delay of Arrival (TDOA) of the sound signals according to angles of the sound signals and geometric relationship formed by the array of the sound receiving portions 10 through Generalized Cross Correlation (GCC in short), adaptive eigenvalue decomposition algorithm (AEDA in short), or Blind Beamforming approach, then adjusts and overlaps the sound signals on the elapsing time axis to intensify the characteristics of the sound signals that contain N1 characteristic values.

Step S4: Retrieve N2 characteristic values of greater impact from the N1 characteristic values of the intensified sound signal, and N2 is smaller than N1. In this embodiment ranking of the N1 characteristic values applicable to disease judgment is processed according to a method presented by F. Chang and J. -C. Chen in the 2010 Conference on Technologies and Applications of Artificial Intelligence, November 2010 entitled “An adaptive multiple feature subset method for feature ranking and selection”, and N2 characteristic values that have greater impact are obtained. The method disclosed in that article also is incorporated in this invention to become a part thereof.

Step S5: Judge the N2 characteristic values matching disease sound signal data in the data portion 40; then the output portion 50 outputs a disease judgment result. In this embodiment the processing portion 30 employs a Support Vector Machine (SVM in short, disclosed by Vladimir Vapnik in 1963, generally deemed one of the best learning modules in terms of pattern recognition capability) as the index of margin, and chooses maximum linear classifier or soft margin classifier as the separation method to classify the N2 characteristic values. In the event that the aforesaid classification methods are not applicable, a generally called “Kernel trick” technique is used to do classification. Then the classified N2 characteristic values are matched against the disease sound signal data in the data portion 40 to get the disease judgment result. Finally, the disease judgment result is output through the output portion 50, such as displaying on a screen (not shown in the drawings).

Also referring to Table 1 below that shows the test outcomes via the Blind beamforming method to intensify and detect the sound signals of patient's lungs. In this test the sound signals issued from the patient's lungs are divided into three types: normal, crackle and wheeze. It is to be noted that according to the aforesaid diseases diagnostic method, at step S3 the Blind beamforming method is chosen to adjust and overlap the received sound signals on the elapsing time axis to intensify the characteristics of the sound signals. At the final step S5, the judgment result of the disease is obtained. The accuracy of detection outcome reaches 85% for the normal sound signal type, 80% for the crackle type, and 90% for the wheeze type. The average accuracy of the three types is 85%.

TABLE 1
Accuracy of detection of intensified sound signals of
lungs by adopting the Blind beamforming method.
Blind beamforming method

| | Detection outcome |
|---|---|
| Lung sound signal type | accuracy |
| normal | 85% |
| crackle | 80% |
| wheeze | 90% |
| Three types (total) | 85% |

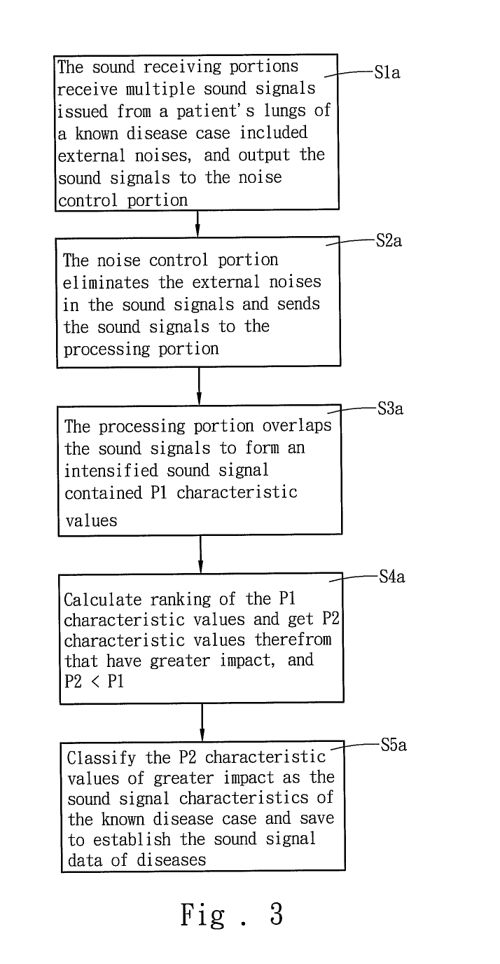

Please refer to FIG. 3 for the process of establishing the disease sound signal data according to the first embodiment. It includes the steps as follows:

Step S1a: Arrange the sound receiving portions 10 in an array fashion to receive sound signals issued from varying location of the lungs 2 of a known disease case, including external noises, and output the sound signals to the noise control portion 20.

Step S2a: The noise control portion 20 generates a counter noise signal according to the external noises to eliminate the external noises, and sends the resulting sound signals to the processing portion 30.

Step S3a: The processing portion 30 receives the sound signals and calculates the time delay of arrival (TDOA) of the sound signals according to angles of the sound signals and geometric relationship formed by the array of the sound receiving portions 10, and adjusts and overlaps the sound signals on the elapsing time axis to intensify sound signal characteristics to form an intensified sound signal containing P1 characteristic values.

Step S4a: Calculate ranking of the P1 characteristic values on disease interpretation, and get P2 characteristic values therefrom that have greater impact, where P2 is smaller than P1. Step S5a: Classify the P2 characteristic values of greater impact as the sound signal characteristics of the known disease case and save to establish the sound signal data of diseases.

As a conclusion, the invention receives the sound signals issued from a patient's lungs through the electronic stethoscope; noises are eliminated from the sound signals, and the sound signals are overlapped and intensified, then characteristics of the sound signal are sampled, compared and interpreted, finally a disease judgment result is output. Thus automatic interpretation of diseases can be accomplished via the electronic stethoscope to reduce human erroneous judgment. Moreover, users can get preliminary understanding of their bodies through the electronic stethoscope, then get comparison and confirmation from the doctors to enhance accuracy of disease interpretation, and get proper treatments as desired. It provides a significant improvement over the conventional techniques.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A method for diagnosis of diseases through an electronic stethoscope which includes at least two sound receiving portions, a noise control portion connected to the sound receiving portions, a processing portion connected to the noise control portion, a data portion connected to the processing portion and contained disease sound signal data and an output portion connected to the processing portion, the method comprising the steps of:
S1: receiving multiple sound signals issued from a patient's lungs included external noises through the sound receiving portions and sending the sound signals to the noise control portion;
S2: eliminating the external noises of the sound signals through the noise control portion and sending the resulting sound signals to the processing portion;
S3: overlapping the sound signals through the processing portion to form an intensified sound signal contained N1 characteristic values;
S4: retrieving N2 characteristic values of greater impact from the N1 characteristic values of the intensified sound signal, wherein N2 is smaller than N1; and
S5: judging that the N2 characteristic values matching the disease sound signal data in the data portion and outputting a disease judgment result through the output portion.

2. The method of claim 1, wherein the step S2 further includes generating a counter-noise signal by the noise control portion to eliminate the external noises.

3. The method of claim 2, wherein the noise control portion includes a sensor to detect the external noises based on which to generate the counter-noise signal.

4. The method of claim 1, wherein the step S3 further includes aligning and overlapping the sound signals on an elapsing time axis by the processing portion after calculation of time delay of arrival.

5. The method of claim 4, wherein the calculation of time delay of arrival is selected from a group of methods consisting of generalized cross correlation, adaptive eigenvalue decomposition algorithm and blind beamforming.

6. The method of claim 1, wherein the step S4 of retrieving N2 characteristic values that have greater impact is performed by calculating the ranking of the N1 characteristic values on disease judgment.
